(19) United States
(12) Patent Application Publication
(10) Pub. No.: US 2012/0115210 A1
(43) Pub. Date: May 10, 2012

(54) OUTDOOR CULTIVATOR FOR PHOTOSYNTHETIC MICROORGANISMS

(75) Inventors: Jared L. Winters, Mesa, AZ (US); Elliot R. Hobbs, Phoenix, AZ (US); John D.V. Winters, Mesa, AZ (US); Ernesto A. Tom, Laveen, AZ (US); Christopher Wright, Chandler, AZ (US)

(73) Assignee: Element Cleantech Inc., Newark, DE (US)

(21) Appl. No.: 13/256,599

(22) PCT Filed: Mar. 29, 2010
(86) PCT No.: PCT/US10/29055

§ 371 Date: Nov. 2, 2011

Related U.S. Application Data
(60) Provisional application No. 61/164,446, filed on Mar. 29, 2009.
(60) Provisional application No. 61/178,441, filed on May 14, 2009.

(51) Int. Cl. C12M 1/42; C12N 1/12

(52) U.S. Cl. 435/257.1; 435/292.1

(57) ABSTRACT

A novel waterborne cultivator, which provides the benefits of known systems for culture of photosynthetic micro-organisms in a novel configuration, incorporating a simplified, two-phase rotary mixing and gas injection system, the two phases being liquid and gas (CO₂). The mixing system provides optimum growth conditions through increased turbulent vertical mixing and increased levels of dissolved CO₂ throughout the cultivator via injection of flue gas or other CO₂-bearing gas stream. The system thus provides efficient capture and sequestration or reuse of CO₂ while producing valuable biomass for food, feed, and fuel use. The waterborne configuration provides further benefits of passive temperature control and automatic leveling for consistent culture depth. Additional benefit is provided by an enclosed design which reduces contamination and evaporative loss by isolating the photosynthetic culture from the outside environment. The simplified and well-integrated design of the cultivator and mixing system greatly reduces capital and operating costs compared to previously known systems.

### CROSS-REFERENCE TO RELATED APPLICATIONS

The present application claims the benefit of U.S. Provisional Application No. 61/164,446, filed on Mar. 29, 2009 and U.S. Provisional Application No. 61/178,441, filed on May 14, 2009, the entire disclosures of both of which are hereby incorporated herein by reference in their entirety.

### FIELD OF THE INVENTION

The present invention relates to an outdoor cultivation system for photosynthetic microorganisms and more particularly, the present invention relates to a system for cultivating phototrophic microalgae in liquid culture through efficient gas and nutrient injection and distribution, and increasing vertical mixing within the liquid culture.

### BACKGROUND

Many types of photosynthetic micro-organisms have been cultivated to produce a wide variety of biomass products, ranging from dietary supplements such as omega-3 fatty acids to aquaculture feed. Recently, as a result of heightened interest in CO₂ sequestration, attempts have been made to grow algae biomass as a source of fuel. For many years algae cultures have been grown outdoors in open ponds, and recently many new bioreactor designs have been developed in order to improve the productivity of algae culturing systems. The present invention combines benefits of several pre-existing designs in combination with novel developments to provide a simplified, low cost scalable system providing ideal growth conditions for algae culture, enabling competitive production of biomass and CO₂ sequestration at a globally significant scale.

The terms “bioreactor”, “reactor” and “cultivator” are used herein interchangeably.

Raceway ponds are currently the most widely used large-scale algae culturing system. Raceway ponds are open ponds, described, for example, by Chaumont, in “Biotechnology of algal biomass production: a review of systems for outdoor mass culture,” Journal of Applied Phycology (1993) 5: 593-604.

Raceway ponds are rectangular systems that consist of two long troughs or “raceways” fluidically connected at either end, through which culture, 20-30 cm deep, is circulated using a paddle wheel at a rate of around 15 cm/sec, to create turbulent conditions. CO₂ is usually injected into a sump at a single point, and the dissolved CO₂ is then circulated with the liquid culture. However, since the pond is open, CO₂ availability can limit the algal growth, and much of the injected CO₂ is lost to the atmosphere. Uptake of the dissolved CO₂ can be increased by making the sump deeper and thereby allowing increased contact time. But making the sump deeper requires increased pumping energy to deliver the gas against the increased hydrostatic head, rendering it impractical for low-concentration CO₂ sources and low-value biomass products. In addition, since raceway ponds systems are often placed in dry sunny regions for high biomass productivity, water loss from the culture due to evaporation from the open culture can be very significant. The open nature of the system also renders the system vulnerable to contamination by other species of algae, predatory microorganisms, and zooplankton, which constantly threaten the optimal algae biomass productivity in raceway ponds systems and thereby limits commercial culture to a few robust species (see “Commercial production of microalgae: ponds, tanks, tubes and fermenters”, by Borowitzka, Journal of Biotechnology, Volume 70, 1999 (“Borowitzka 1999”); and “Photobioreactors: production systems for phototrophic microorganisms”, by Pulz, Applied Microbiology and Biotechnology, Volume 57, 2004). Additionally, practical constraints such as providing sufficient thermal mass to maintain desired temperature during the day/night thermal cycle, and providing sufficient depth to guarantee turbulent flow throughout the entire raceway, typically dictate a minimum culture depth in raceways that is greater than would be biologically optimum for common light tolerant species (see “Handbook of Microalgal Culture”, Blackwell Publishing, Richmond ed., 2004). Therefore, the maximum culture density of raceway is effectively limited, increasing the cost of biomass extraction. Finally, earth-moving equipment to produce a flat level surface for the masonry construction of the pond requires a significant expense. Regardless of the disadvantages of the raceway pond, the raceway pond has proven to be a simple and efficient design for culturing algae on a large scale, and has provided information on beneficial large scale culturing conditions for many microalgae species.

Another widely used large scale algae-pond design is that of circular ponds mixed by a single, centrally pivoted agitating arm (see Borowitzka 1999). Circular ponds systems have been used to grow microalgae species such as chlorella for many years (see “Microalgal mass culture systems and methods: Their limitation and potential”, by Lee, Journal of Applied Phycology, (2001) 13(4):1573-1576). Circular ponds require expensive concrete construction, and similarly to the raceway, often inject CO₂ at only a single point and rely on the mechanical mixing of a single mixing arm rotating through the pond. As a result, although circular ponds have the ability to consistently grow algae, productivity would benefit from more extensive mixing and better distribution of CO₂ injection.

An established practice employing a different version of the circular pond has been used in the Ukraine as well (“Ukraine-circular-pond”). A Ukraine-circular-pond utilizes smaller diameter circular ponds constructed from metal rather than concrete. The mixing in Ukraine-circular-ponds is facilitated by a single centrally attached, circulating, submerged mixing arm. Like in the other prior art systems, the Ukraine-circular-pond design utilizes sparging of CO₂ at a single point in the system, separate from the processes of mechanical mixing.

Systems for culturing microorganisms have also been deployed at the surface of bodies of water. For example, U.S. Pat. No. 3,955,317, given to Claude Gudin, provides a system which includes a raft of elongated tubes of transparent polyethylene film containing a mixture of water and gas, floating on the ocean surface or other pool of water. Advantages of the arrangement include semi-passive temperature control, reduced structural loads permitting the use of inexpensive containment materials, and automatic leveling of the culturing system, which facilitates circulation.

Although a variety of systems have been devised for culturing photosynthetic microorganisms, none has achieved the combination of low cost, ease of operation, robustness, and scalability to allow large-scale cultivation at globally significant scale. There is therefore a need and it would be beneficial to have a microorganism cultivator for photosynthetic species that provides substantially ideal growth conditions for useful organisms at high culture density, while sequestering CO₂ from a waste gas stream. It would be further beneficial for such a system to be enclosed to prevent contamination and loss of culture volume to evaporation. It would be further beneficial for such a system to be operable in regions not suitable for conventional crops. It would be further beneficial for the system to be inexpensive, mechanically robust, and suitable for integration into scalable biomass production systems up to and beyond 10 km² in size.

### SUMMARY OF THE INVENTION

According to the teachings of the present invention, there is provided a novel waterborne photosynthetic cultivator, which provides the benefits of known systems for culture of photosynthetic organisms in a novel configuration. Depending on their size and number floating on a body of water, the waterborne cultivators of the invention find use in the large scale and seed scale culture of photosynthetic microorganisms. Floating the cultivators on a host pool of water allows for improved thermal control of the culture fluid within the cultivator. In particular, the water of the host pool underneath the floating cultivators counteracts overheating of the culture fluid inside the cultivator.

The photosynthetic cultivator includes a simplified, two-phase rotary mixing and gas injection system, the two phases being liquid and gas (CO₂). The mixing system provides optimum growth conditions through increased turbulent vertical mixing and increased levels of dissolved CO₂ throughout the cultivator via injection of flue gas or other CO₂-bearing gas stream. The system thus provides efficient capture and sequestration or reuse of CO₂ while producing valuable biomass for food, feed, and fuel use.

The waterborne configuration provides further benefits of passive temperature control and automatic leveling for consistent culture depth.

An aspect of the present invention is to provide an enclosed design which reduces contamination and evaporative loss by isolating the photosynthetic culture from the outside environment.

The simplified and well-integrated design of the cultivator and mixing system greatly reduces capital and operating costs compared to previously known systems. The floating cultivators of the invention utilize naturally occurring sunlight, rather than artificial light, for the cultivation of photosynthetic microorganisms in a horizontal plane. The pumps driving the co-circulation of the CO₂ and microorganisms can be powered by generators or turbines connected to renewable energy sources, e.g., solar, wind or wave energy sources, as well as derive power from non-renewable energy sources.

Accordingly, in one aspect, the invention provides waterborne cultivators for photosynthetic micro-organisms, comprising:
a buoyant frame connected to a top sheet sufficiently transparent to admit light to support photosynthetic growth of the micro-organisms and a bottom sheet, the top sheet and bottom sheet connected to a central hub assembly in an arrangement to create a cultivation space comprising culture fluid comprising a substantially homogenous population of photosynthetic micro-organisms, the central hub assembly comprised of a vertical hollow cylinder forming a vertical axis, the cylinder having an inlet and one or more outlets in fluid communication with one or more mixing wands extending substantially perpendicular from the cylinder, the mixing wands having one or more or more holes on one side of the wand, wherein the culture fluid is delivered into the inlet and through the holes of the mixing wands, wherein the wands rotate with the central hub assembly around the vertical axis, wherein fluid pumped through the wands is ejected into the culture fluid, thereby promoting turbulent vertical mixing of the photosynthetic micro-organisms.

In a further aspect, the invention provides a cultivation system for photosynthetic micro-organisms, comprising:
a buoyant frame connected to a top sheet sufficiently transparent to admit light to support photosynthetic growth of the micro-organisms and a bottom sheet, the top sheet and bottom sheet connected to a central hub assembly in an arrangement to create a cultivation space comprising culture fluid comprising a substantially homogenous population of photosynthetic micro-organisms, the central hub assembly comprised of a vertical hollow cylinder forming a vertical axis, the cylinder having an inlet and one or more outlets in fluid communication with one or more mixing wands extending substantially perpendicular from the cylinder, wherein the mixing wands have one or more or more holes on one side of the wand;
a recirculation pump in fluid communication with the culture fluid, wherein the pump draws culture fluid from the cultivation space through a suction line and returns culture fluid to the cultivation space via a pressure line into the inlet and through the holes of the mixing wands, wherein the wands rotate with the central hub assembly around the vertical axis, wherein culture fluid pumped through the wands is ejected into the culture fluid in the cultivation space, thereby promoting turbulent vertical mixing of the photosynthetic micro-organisms. The systems can further comprise an external CO₂ source in communication with the recirculating culture fluid; one or more pH monitors in communication with the recirculating culture fluid; an air inlet in the top sheet to deliver external air into the cultivation space; an air outlet in the top sheet to discharge oxygen-rich air from the cultivation space; blowers in communication with the air inlet and/or air outlet driving air circulation of the cultivation space, and/or a host pool, as described herein.

With respect to the embodiments of the cultivators, in some embodiments, the cultivators are floating on a host pool. The host pool can be partially or entirely man-made or naturally occurring. The host pool can be outside, not indoors.

In some embodiments, the photosynthetic micro-organism is phototrophic algae. In some embodiments, the microalgae in the culture are Selenestrum, Scenedesmus, Nannochloropsis or Isochrysis.

In some embodiments, the bottom sheet and top sheet connected to the frame and the central hub assembly form a liquid-tight enclosure. The enclosure allows for gas exchange and the discharge of oxygen. For example, in some embodiments, the top sheet has an air inlet for intake of air external to the cultivation space and an air outlet for discharge of oxygen-rich air from within the cultivation space.

In some embodiments, the culture fluid is in fluid communication with an external source of CO₂. In some embodiments, CO₂ is co-delivered into the inlet with the culture fluid.

In some embodiments, the inlet of the cylinder is in fluid communication with a pump. In some embodiments, the pump is in fluid communication with an external source of CO₂ and at least one pH monitoring unit. Culture fluid is recirculated from the culture space of the cultivator to the pump and returned to the culture space. In some embodiments, CO₂ from the external source is delivered into the recirculating culture fluid prior to processing through the pump. In some embodiments, CO₂ from the external source is delivered into the recirculating culture fluid after processing through the pump. At least one of the pH monitors are in communication with the recirculating culture fluid. pH can be measured prior to and/or after CO₂ injection into the recirculating culture fluid stream. If the pH measured prior to and/or after CO₂ delivery is above the target pH value, CO₂ is injected into the recirculating culture fluid. If the pH measured prior to and/or after CO₂ delivery is at or below the target pH value, CO₂ is not injected into the recirculating culture fluid.

In some embodiments, the inlet of the cylinder is in fluid communication with a mixer, e.g., for mixing the CO₂ with recirculating microorganisms before redelivering to the cultivation space.

In some embodiments, the fluid pumped through the wands creates a reaction force which rotates the central hub assembly.

In some embodiments, the inlet of the cylinder is in fluid communication with a motor and impeller integrated into the central hub assembly.

In some embodiments, a motor turns the central hub assembly.

In some embodiments, the central hub assembly is in fluid communication with a processer that monitors the culture fluid conditions.

In some embodiments, the inlet of the cylinder is submerged in the culture fluid. In some embodiments, the mixing wands are submerged in the culture fluid.

In some embodiments, the top sheet is raised above the bottom sheet by inflation, e.g., to create a dome maintained by internal air pressure. In some embodiments, the top sheet has an air inlet and an air outlet, wherein fresh air is delivered into the cultivation space via the air inlet and oxygen-rich air is discharged outside the cultivation space via the air outlet. The air inlet and the air outlet can be in communication with pipes, ducts or tubing for delivery of fresh air into the cultivation space and discharge of oxygen-rich air out of the cultivation space. In some embodiments, the flow of the fresh air delivered into the cultivation space and the oxygen-rich air discharged from the cultivation space is facilitated or governed by a blower or fan in communication with the inlet and/or outlet tubing, pipes or ducts.

In some embodiments, the top sheet is supported by an internal frame.

In some embodiments, the frame is circular. In some embodiments, two or more ratchet mechanisms are attached to the frame for attachment to supporting straps, e.g., over the top sheet and/or under the bottom sheet.

In some embodiments, the culture fluid comprises substantially uniformly distributed CO₂.

In some embodiments, the photosynthetic microorganisms are grown in the cultivators of the invention without artificial light, i.e., only in the presence of naturally occurring solar radiation. In some embodiments, the photosynthetic microorganisms are grown in a horizontal plane, e.g., not in a vertical tube.

In a related aspect, the invention provides a culture fluid produced in a cultivator of the present invention, comprising a homogenous population of photosynthetic algae cells, wherein the culture fluid comprises substantially uniformly distributed CO₂ and the individual algae cells in the population are or have been substantially uniformly exposed to light.

In a further aspect, the invention provides methods for cultivating photosynthetic microorganisms by culturing the photosynthetic microorganisms in a waterborne or floating cultivator as described herein. The methods allow for culturing photosynthetic microorganisms in a culture fluid comprising substantially uniformly distributed CO₂. The culture fluid is further maintained at a stable temperature. Further embodiments of the methods are as described herein.

In a further aspect, the invention provides a host pool comprising one or more of the floating photosynthetic microorganism cultivators described herein. The host pool can be partially or entirely man-made or naturally occurring.

Further embodiments of the invention are described herein.

### BRIEF DESCRIPTION OF THE DRAWINGS

The present invention will become fully understood from the detailed description given herein below and the accompanying drawings, which are given by way of illustration and example only and thus not limitative of the present invention, and wherein:

FIG. 1 is a schematic illustration of a photosynthetic cultivator module, according to embodiments of the present invention;

FIG. 2A is a side view illustration of a preferred configuration of cultivator module, according to embodiments of the present invention;

FIG. 2B is a top view illustration of a preferred configuration of cultivator module, according to embodiments of the present invention;

FIG. 3A illustrates a detail view in cross section of the central hub assembly and a portion of the mixing assembly of the cultivator module shown in FIG. 2A;

FIG. 3B illustrates a detail of the central hub assembly.

FIG. 4A illustrates a variation of the frame of the cultivator module shown in FIG. 2A, in cross section, designed for volume manufacturing configuration;

FIG. 4B illustrates other embodiments of a bottom pre-fabricated sheet assembly, according to variations of the present invention;

FIG. 5 illustrates another embodiment of a photosynthetic cultivator module, according to variations of the present invention;

FIG. 6 illustrates another embodiment of a photosynthetic cultivator module, according to variations of the present invention, designed for shoreside applications; and

FIG. 7 illustrates another embodiment of a photosynthetic cultivator system, according to variations of the present invention, designed for shoreside applications.

FIG. 8 illustrates an embodiment of the floating cultivator floating in an above-ground pool.

FIG. 9 illustrates the flow path for the culture fluid recirculation (depicted as dotted lines).

FIG. 10 illustrates the initial inflation of the upper canopy in embodiments where the dome of the upper canopy is maintained by internal air pressure.

FIG. 11 illustrates a balance between the fresh exterior air supplied to the canopy and the air that discharges, once the upper canopy is raised by air inflation.

### DESCRIPTION OF THE PREFERRED EMBODIMENTS

Before explaining embodiments of the invention in detail, it is to be understood that the invention is not limited in its application to the details of construction and the arrangement of the components set forth in the host description or illustrated in the drawings.

Unless otherwise defined, all technical and scientific terms used herein have the same meaning as commonly understood by one of ordinary skill in the art to which the invention belongs. The methods and examples provided herein are illustrative only and not intended to be limiting.

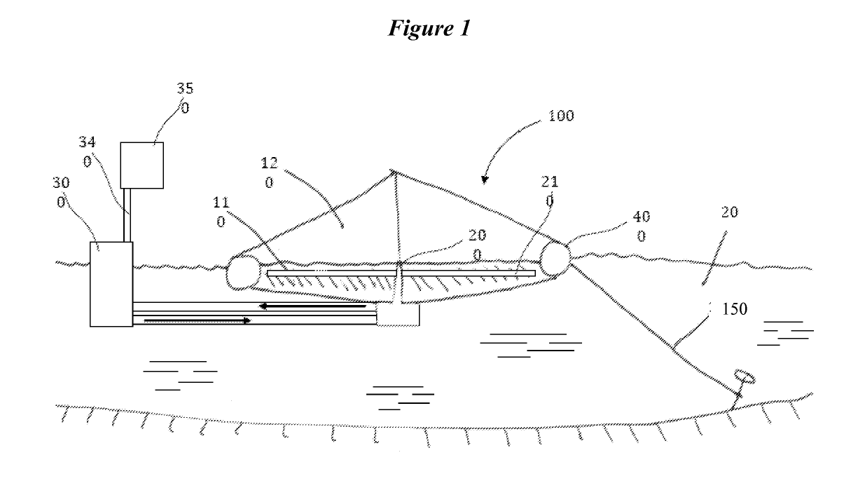

Reference is now made to FIG. 1, which is a schematic illustration of photosynthetic cultivator module 100, according to embodiments of the present invention. A cultivator system of the present invention may include of one or more photosynthetic cultivator modules 100 floating on host water pool 20 and containing photosynthetic culture fluid (i.e., comprising water, minerals, photosynthetic micro-organisms, CO₂) 110 and gas 120. The culture fluid within the cultivator is maintained at a depth such that the water level is approximately the same as the water level outside the cultivator (i.e., in the host pool) and sufficiently high to completely submerge the mixing wands 110 and 210. For example, the culture fluid can be maintained at a depth in the range of about 5-30 cm, for example, 10-30 cm or 20-30 cm, for example, about 5 cm, 10 cm, 15 cm, 20 cm, 25 cm or 30 cm. Depending on the diameter (or radius) of the cultivator and the distance from the cultivator to the recirculating pump, the volume of culture fluid can be in the range of about 10,000 L to about 100,000 L, for example in the range of about 10,000 L to about 50,000 L, for example, about 10,000 L, 12,000 L, 15,000 L, 20,000 L, 25,000 L, 30,000 L, 40,000 L, 50,000 L, 60,000 L, 70,000 L, 80,000 L, 90,000 L or 100,000 L culture fluid.

One or more photosynthetic cultivator modules are floated in a host water pool. The host water pools can be entirely or partially man-made or naturally occurring. The host pool can be above or below ground. The host pools are of a sufficient volume and depth to allow the one more photosynthetic cultivator modules to freely float without touching the bottom surface. In certain embodiments, waters of a greater depth are preferable to facilitate better thermal control or maintenance of a stable temperature of the culture fluid. Preferably, the host pool does not subject the cultivators to high wave action or turbulent waters that may damage the cultivators and unduly agitate the culture fluid. Preferably, the host pool also has circulating currents to facilitate stable temperature maintenance of the culture fluid within the cultivator. There is an advantage in mounting the cultivators in a host water pool having slight currents. In the presence of high solar radiation and little circulation under the cultivator, the exterior water, which cools the culture fluid, stagnates. This causes heating of the culture fluid. If there is little current, the heat is not effectively removed and, in turn, the temperature of the cultivator rises. In the presence of current, this effect is eliminated. For example, it is envisioned that a series of two or more photosynthetic cultivators could be placed in a constructed channel allowing slow flow of a natural water source or, a man-made water source, e.g., associated with the existing site operations. The water flowing in the channel constantly refreshes of the water underneath the cultivators, thereby allowing for thermal control or maintenance of a stable temperature of the culture fluid.

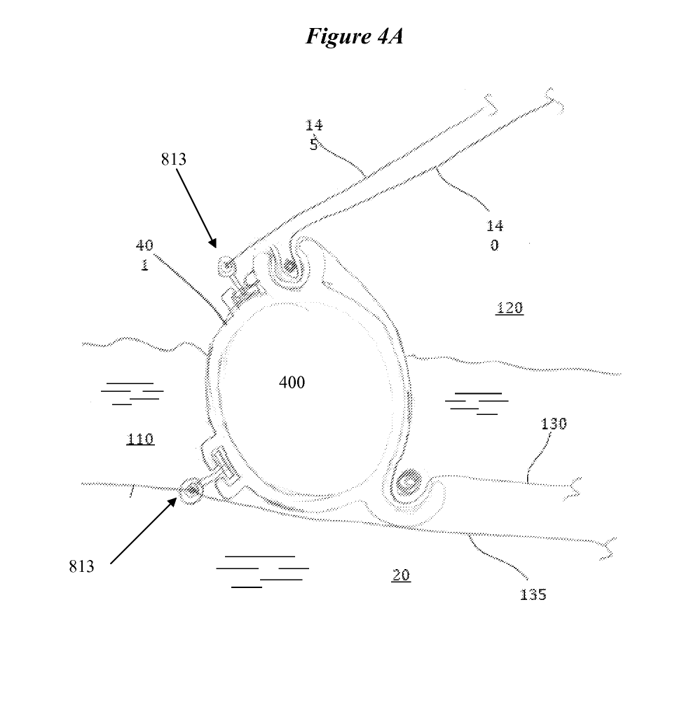

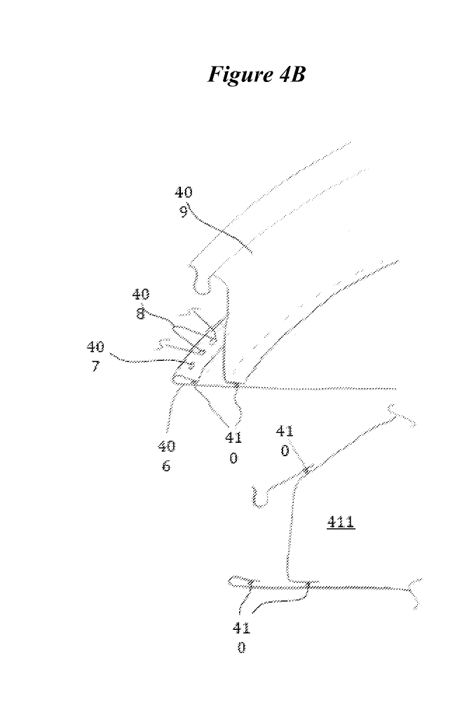

Another scenario envisions anchoring the cultivators in a natural bay or within a breakwater. Alternatively, the cultivators could be located at an unprotected oceanside site, for example, with a land based man-made pond system that could be filled with ocean water on a regular basis to support the floating cultivators in the man-made lake or lagoon. In other embodiments, the cultivators can be floated on a man-made float pond, wherein the water provides level support for floating the cultivators, rather than having to ensure that the ground is graded properly, reducing construction and site preparation costs. Preferably, photosynthetic cultivator module 100 further includes anchoring sub system 150 (FIG. 4A) that holds cultivator module 100 in place. Associated support systems 300 provide inputs and to cultivator module 100. In some embodiments, an array of two or more cultivator modules are anchored together in a host water pool. For example, multiple cultivator units could be lashed together and anchored in a sheltered cove on a naturally occurring body of water. An individual pump can recirculate the culture fluid through one or more cultivator modules, for example, 1, 2, 3, or more, cultivator modules.

Further salient features of the floating cultivators include culturing the photosynthetic microorganisms in a horizontal plane rather than vertical tubes. Also, the cultivators are for use outdoors and rely on natural solar radiation rather than artificial light for the growth of the photosynthetic microorganisms. The cultivators are not air-tight in order to allow for circulation of gas, e.g., delivery of CO₂ into the cultivation space and discharge of O₂ from the cultivation space. Carbon dioxide delivery or sparging into the culture fluid occurs upstream of the growth site, i.e., during the recirculation process, rather than downstream of the growth site or within the cultivation space. The photosynthetic microorganisms are recirculated and processed through a system that monitors culture conditions, e.g., pH. CO₂ is delivered into the recirculating culture fluid stream if the culture pH rises above a predetermined high pH threshold value and not delivered into the recirculating culture fluid stream if the culture pH falls below a predetermined low pH threshold value. The photosynthetic microorganisms in the culture fluid are subject to vertical agitation and the CO₂ is evenly mixed throughout the culture fluid by simultaneous jetting of recirculated culture fluid and sparged CO₂ through holes in mixing wands submerged in the culture fluid. The jetting of the mixture of recirculated culture fluid and sparged CO₂ causes the mixing wands to turn around the axis of the central hub in the cultivator. The pumps driving the recirculation of the culture fluid can be powered by renewable energy sources.

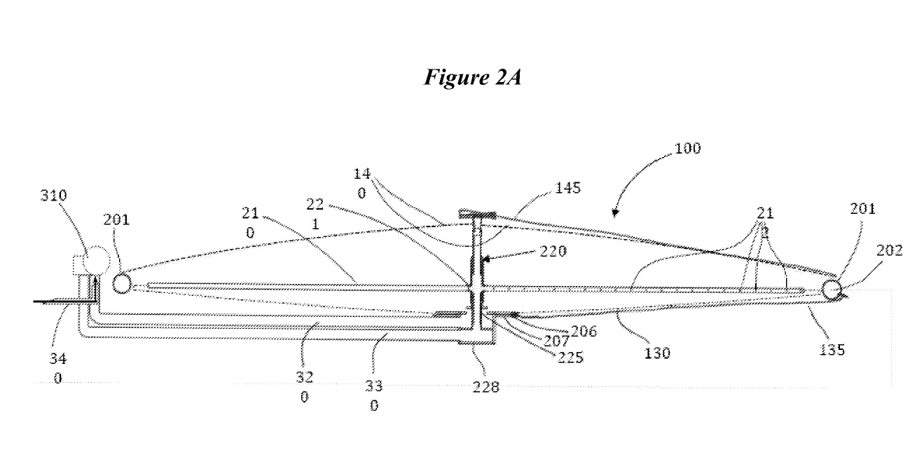

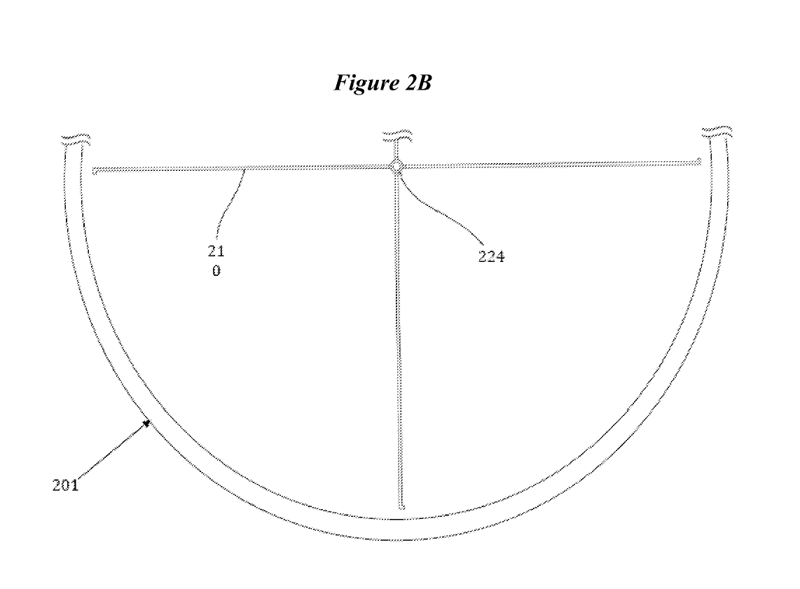

FIGS. 2A and 2B show the components of a preferred configuration of cultivator module 100, whereas FIG. 2a is a side view and FIG. 2b is a top view of cultivator module 100. Buoyant semi-rigid frame 400 (depicted, e.g., in FIGS. 1 and 4A) of circular cross-section tube/pipe 201 is preferably formed into a ring. Frame 400 is described herein as having a ring shape, but with no limitation on any other shape of frame 400 and the cross-section of frame 400. Ring 400 can be formed in any diameter sufficient to support the floating of the cultivator. As desired, the cultivators can have a diameter of at least about 5 m, for example, in the range of about 5-50 m, or 10-20 m, for example about 5 m, 10 m, 12 m, 15 m, 20 m, 25 m, 30 m, 35 m, 40 m, 45 m or 50 m. Ring may be formed of plastic, metal, fiberglass, with UV-inhibited polyethylene as a good choice. Frame 400 may be filled with closed cell marine polyurethane foam 202 to ensure long-term buoyancy and to prevent leaks.

The cultivator module 100 further includes mixing assembly 200. Radial bottom straps 135 (FIG. 2A), made of, for example, from woven polymer fiber or any other suitable non-corroding, non-abrasive material are installed and tensioned between frame 400 and bottom 224 of central hub assembly 220. Additional straps transecting the circular profile cultivator chordally or concentrically may also be installed and tensioned. Alternatively, a net or mesh may be stretched into place. A sheet of reinforced polymer film, for instance, commercially available polyethylene film or fiber-reinforced pond liner such as MFG/PRODUCT, is laid into reactor 100 to form bottom sheet 130. Depending on the specific culture conditions, bottom sheet 130 might be white (to reflect transmitted light back to the culture), clear (to allow transmitted light to escape) or dark (to convert transmitted light to heat, as may be desirable in colder climates). Bottom sheet 130 is attached to the circular frame 400 by suitable sealing means as are well known in the agricultural greenhouse industry. For example, gasket seal 206 is also disposed between bottom sheet 130 and a flange 207 on central hub assembly 220 to complete a liquid-tight enclosure. Top sheet 140 of transparent or semi-transparent flexible polymer film or reinforced polymer film is positioned over top plate 222 of central hub assembly 220 and attached to frame 400, effectively sealing the enclosure to prevent contamination and release of gas 120. A second set of radial straps 145 extends from a second set of ratcheting tensioners (813) upward to attachment points at top plate 222 of the central hub assembly 400.

A plurality of mixing wands 210 fabricated, for example, from standard PVC pipe or other suitable non-corrosive neutrally-buoyant material, are threaded into fittings 211 welded to central hub assembly 400 just below the surface of culture 110. The wands are usually arranged in diametrically opposed pairs, but odd numbers of wands may also be attached. The number of wands extending from the central hub assembly will depend on the thickness or diameter of the central hub assembly. For example, 2-8 radial mixing wand may extend from the central hub assembly. In some embodiments, 2, 3, 4, 5, 6, 7, 8 wands extend from the central hub assembly. In some embodiments 4 mixing wand extend from the central hub assembly. The wands can, but need, not be placed at the same height on the central hub, but are placed at a height that is below the surface of the culture fluid so that the wands are submerged in the culture fluid.

The wands 210 are pierced at intervals by nozzles or holes 212 on one side, such that fluid pumped through the wands is ejected tangentially, creating a reaction force which rotates central hub assembly 400. Nozzles 212 may be spaced at alternating radial positions on adjacent wands, and may be directed at an angle with respect to the tangent sufficient to propel the wands while concurrently scouring or agitating microorganisms that have settled on the bottom sheet 130, lifting or stirring the microorganisms back into the culture fluid. For example, the nozzles or holes in the mixing wands can be aimed at an angle that is in the range of about 30° to about 60° below the surface of the culture medium, for example, at an angle in the range of about 40°-50° below the surface of the culture medium, for example, at an angle that is about 30°, 35°, 40°, 45°, 45°, 50°, 55° or 60° below the surface of the culture medium. The mixing wands usually extend the length of the cultivator module, e.g., the length of the radius where the cultivator is circular. As appropriate or desired, the nozzles can be spaced at 0.1 m, 0.2 m, 0.3 m. 0.4 m, 0.5 m, or smaller or larger, intervals along the mixing wands. The nozzles can, but need not be, evenly spaced on a mixing wand. In order to maximize vertical mixing, the nozzles can be spaced differently along different mixing wands. The holes or nozzles in the wands are large enough in diameter to jet or spray culture fluid without damaging or shearing the microorganisms and small enough in diameter to allow for jetting of the culture fluid that provides sufficient reaction force to propel the mixing wands. In embodiments where the nozzles expel culture fluid in a spray cone, the cone of liquid is usually at a spray angle that is less than about 60°, for example, in the range of about 5° to about 60°, for example, a cone with a spray angle of about 5°, 10°, 15°, 20°, 30°, 40°, 50° or 60°, as appropriate to provide sufficient reaction force to propel the mixing wands.

The mixing action and jetting action of the mixing wands distribute the CO₂ in a substantially homogeneous manner throughout the culture fluid. This intends that concentrations of CO₂ throughout the culture fluid is essentially uniform, for example, at least 90% of the total volume of the culture fluid has a uniform concentration of CO₂, for example, at least 91%, 92%, 93%, 94%, 95%, 96%, 97%, 98% or 99% of the total volume of the culture fluid has a uniform concentration of CO₂. In some embodiments, the distribution of CO₂ in the culture fluid is homogenous or of a uniform concentration throughout the total volume of the culture fluid.

Below bottom sheet 130, central hub assembly 220 connects to pressure line 330 and suction line 320. Lines 320 and 330 connect to the outlet and inlet respectively of to pump 310, for example a centrifugal pump. Lengths of run for the suction and pressure lines (e.g., tubing, ducts or pipes; flexible or rigid) are at least the length of the radius of the cultivator, for example, in cases where the pump is mounted on the frame of the cultivator. The lines are longer in situations where the pump is mounted on a separate platform or on the side of the host pool. The suction and pressure lines are oftentimes about the same length, and are at least the length of the radius of the cultivator. In some embodiments, the suction and pressure lines can be at least about 0.5 to about 10 m, for example, about 0.5 m, 1.0 m, 1.5 m, 2.0 m, 2.5 m, 3.0 m, 3.5 m, 4.0 m, 4.5 m, 5.0 m, 6.0 m, 7.0 m, 8.0 m, 9.0 m, 10.0 m, or longer than the radius of the cultivator. The inlet and outlet pipes and the suction and pressure lines can be about 2.5 cm to about 10 cm in diameter or about 2.5 cm to about 5.0 cm, for example, about 2.5 cm, 3.0 cm, 3.5 cm, 4.0 cm, 4.5 cm, 5.0 cm, 5.5 cm, 6.0 cm, 6.5 cm, 7.0 cm, 7.5 cm, 8.0 cm, 8.5 cm, 9.0 cm, 9.5 cm or 10.0 cm in diameter. Depending on the volume of culture fluid to be recirculated, turnover rate for recirculation of the culture fluid can be about 0.5 hours to about 5 hours, for example, about 0.5 hours to about 2 hours, for example, about 0.5 hr, 1.0 hr, 1.5 hr, 2.0 hr, 2.5 hr, 3.0 hr, 3.5 hr, 4.0 hr, 4.5 hr or 5.0 hr. Smaller volumes of culture fluid will completely recirculate in less time. The mixing wands have a diameter in the range of about 1.0 cm to about 4.0 cm, for example, about 1.0 cm, 1.5 cm, 2.0 cm, 2.5 cm, 3.0 cm, 3.5 cm or 4.0 cm in diameter. Gas containing CO₂ from external source 350 (see FIG. 1) is injected upstream through inlet 340 of centrifugal pump 310, allowing vigorous mixing with culture 110 within the impeller of pump 310. Static mixers may optionally be incorporated in injection line 330 and/or wands 210 to further increase mixing.

As desired or appropriate, the pump or pumps can be mounted on the frame of the cultivator module, on a floating platform dedicated to the pump positioned near the cultivator module(s), or on land abutting the host pool and near the cultivator module(s). Tubing or pipes, e.g., flexible or linear, can be used to connect the suction line 320 and pressure line 330 to the cultivator module. For example, in one scenario, one or more pumps are mounted on a platform (e.g., a dock or raft; the platform can be floating in the host pool or mounted, e.g., to the bottom or a side of the host pool) positioned in a central location in the host pool and one or more cultivator modules connected to the pumps are positioned around the platform with the mounted pumps. The ratio of pump to cultivator can be 1:1, 1:2, 1:3, as appropriate. In some embodiments, a single pump recirculates the culture fluid in one cultivator module. Pumps that find use include Goulds Pump G&L Series SSH Centrifugal Pump, Model 4SH2K52CO with a 7.5 HP, 3Phase TEFC motor. The pumps used can have a variable frequency drive (e.g., a Goulds Aquavar CPC Model CPC401213R). The variable frequency drive enables the pump speed to be increased or decreased, thus, increasing or decreasing the recirculation flow and the rotational speed of the wands within the cultivator. The flow through the recirculation pump is maintained at a speed sufficient to propel the mixing wands to both mix the culture and uniformly distribute the CO₂ in the culture fluid. The flow of culture fluid through the pump and suction/pressure lines can be in the range of about 200 liters per minute (1 pm) to about 800 lpm, or in the range of about 300 lpm to about 500 lpm, depending on the extent of mixing desired to introduce into the culture fluid. In some embodiments, the flow of culture fluid through the pump and suction/pressure lines is about 100 lpm, 150 lpm, 200 lpm, 250 lpm, 300 lpm, 350 lpm, 400 lpm, 450 lpm or 500 lpm, depending on the extent of mixing desired to introduce into the culture fluid. Some species of photosynthetic microorganisms require relatively high culture fluid mixing for growth, while other species of photosynthetic microorganisms require relatively low culture fluid mixing for growth. The linear speed of the mixing wands can be in the range of about 20 cm/sec to about 50 cm/sec, for example, about 20 cm/sec, 25 cm/sec, 30 cm/sec, 35 cm/sec, 40 cm/sec, 45 cm/sec, or 50 cm/sec.

Preferably, an exhaust port is provided at top 222 of central hub assembly 220 to permit depleted gas to escape, wherein the gas may be released or may be piped from cultivator 100 for subsequent processing. In one embodiment, the top or upper sheet of the cultivator is raised, e.g., into a canopy dome, by air inflation. A first blower can be used to supply external air to the vapor space above the culture. A second blower can be used to discharge oxygen-rich air from the cultivation space. The blowers can run continuously to maintain pressure sufficient to inflate the upper sheet or canopy into a dome. Alternatively, the air supply and discharge lines can contain static insulating mechanisms (e.g., air valves) that maintain the internal air pressure within the cultivation space. In such embodiments, the blowers can run intermittently, or on an as-needed basis. In an alternative embodiment, the upper canopy can be supported by an internal frame.

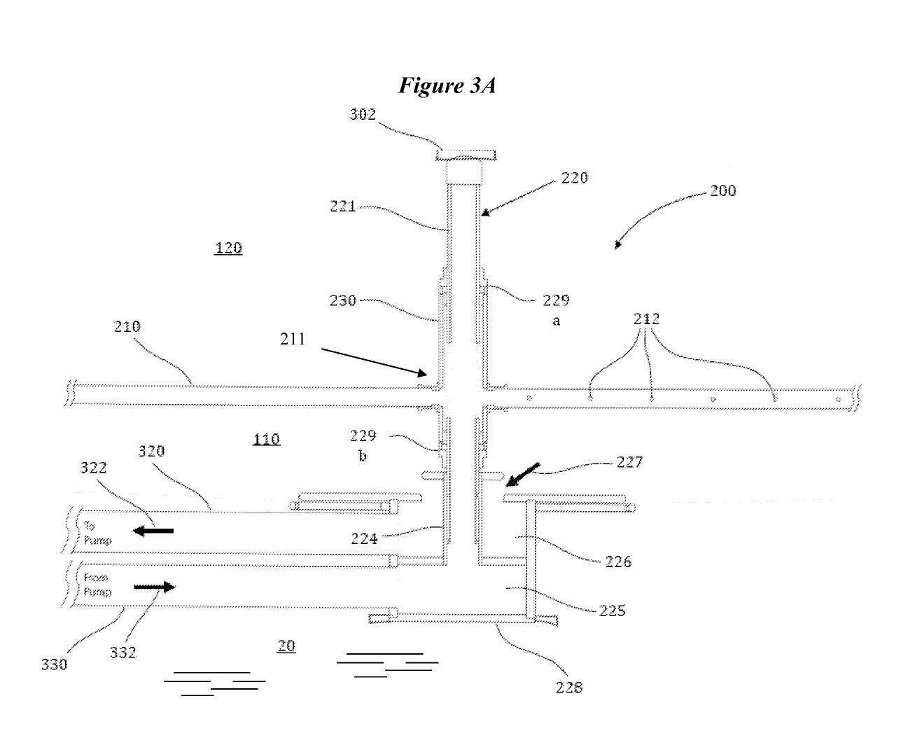

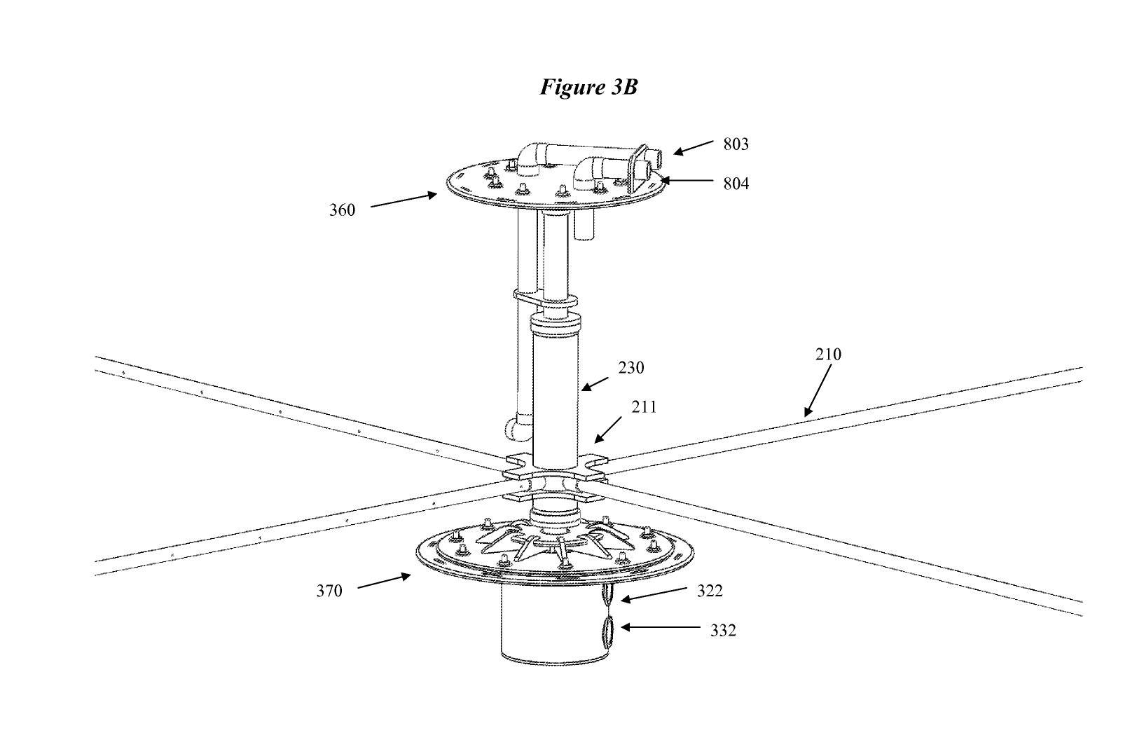

FIG. 3A illustrates a detail view in cross section of central hub assembly 220 and a portion of mixing assembly 200, which consists of stationary and rotating parts, fabricated from PVC plastic or other suitable non-corrosive materials. The stationary part includes bottom plate 228 providing attachment for bottom radial straps 135 (FIG. 2A), a pressure plenum (225), a suction sump (226) communicating with the culture (110) volume via suction intake passage (227), a hollow central axle (221), and upper plate 222 providing attachment for upper radial straps 145. The central axle is hollow and thereby allowing culture 110 to flow into rotating part 230 of hub assembly 220. Rotating part 230 includes fittings 211 affixed thereto, for housing mixing wands 210.

FIG. 3B illustrates a further detailed view of the central hub assembly. The mixing wands (here, four of them) attach to the subassembly that rotates about the central hub (230), thereby comprising the rotary union. The remaining depicted parts are static. The upper canopy air inlet (803) and air discharge (804) ducts are also depicted. The culture fluid inlet (332) and outlet (322) piping connectors located below the hub are shown. The bolts in the lower plate clamp the two plates together (360), thus providing a clamp for the lower film layer. The bolts on the upper plate clamp the two sides together (370) to hold the upper canopy film in place.

Upper and lower commercially available bearing/seal assemblies 229 prevent leakage and wear between the stationary and rotating hub portions. In a preferred mode of operation, motive power for mixing assembly 200 is provided by centrifugal pump 310, which produces fluid flow through wands 210, and thereby operatively rotates central part.

FIG. 4A illustrates a variation of frame 400, in cross section, designed for volume manufacturing configuration. In an embodiment of the invention geared towards volume manufacturing, frame 400 includes a tubular body, attached to a specialized custom extrusion profile 401 incorporating purpose-designed snap-in and/or T-slot features designed specifically for interface with top 140 and bottom sheets 130, tensioning respective top 145 and bottom straps 135, and anchoring sub system 150. The attachment point of bottom sheet 130 to frame 400 may be moved below the free surface of culture 110, to allow a more uniform depth of culture 110 throughout the reactor 100.

In variation of the present invention, ratcheting tensioners (813) are replaced by pre-tensioned straps with sufficient elasticity to maintain tension over time and despite thermal cycling.

Reference is now made to FIG. 4B, which illustrates other embodiments of a bottom pre-fabricated sheet assembly, according to variations of the present invention, Planar bottom sheet 130 is replaced by a pre-fabricated sheet assembly made in the form of a pan incorporating an external peripheral flange 406 provided with grommets 407 or loops permitting attachment via elastic lacing 408, and a separate vertical side wall 409 for culture containment, said side wall being mechanically attached and fluidically sealed by thermal or other welding means 410. Elastic lacing may be replaced by tension springs fabricated from non-corroding material, or other suitable tension-providing elements. This configuration provides secure attachment of the bottom sheet to the frame, and a clean interface between the bottom and the side of the reactor 100, eliminating dark crevices where parasitic organisms may be favored. The separate top and bottom sheets which are installed individually onsite may be replaced by a single factory-made assembly consisting of flexible sheets thermally welded together in the desired rotationally symmetric pod configuration 410. Such a pre-fabricated combined pod may be fabricated in the form of a pan incorporating the peripheral flange attachment system previously described.

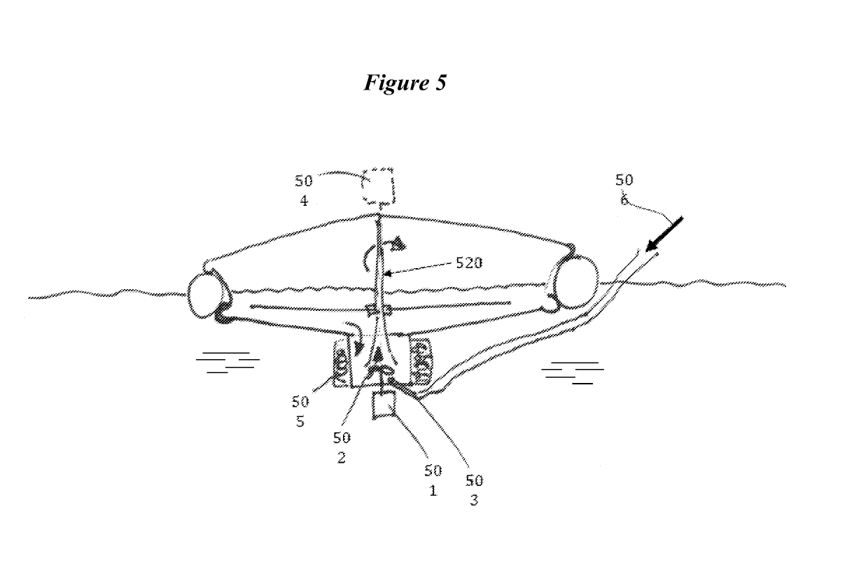

Reference is now made to FIG. 5, which illustrates photosynthetic cultivator modules 500, according to variations of the present invention. External circulation pump 310 connected to central hub assembly 220 by submerged piping is replaced by a motor 501 and impeller 502 integrated into central hub assembly 520 (eliminating the expensive piping and the associated fluidic energy losses). In this case gas is injected through piping means 503 into the central hub just upstream of impeller 502, providing the desired intimate two phase mixing for efficient mass transport. Motor 501 may be installed below central hub assembly 520 to provide optimal cooling; alternatively motor 501 may be installed above the cultivator 500 (location 504) and connected to impeller 502 with a long shaft, facilitating maintenance and replacement. Clearances and impeller loadings may be decreased relative to conventional pump designs to reduce shear-induced damage to the photosynthetic microorganisms. In a preferred mechanical configuration, the vertical position of central hub assembly 520 may be stabilized by an addition of buoyant material 505 below the fluid surface. Although central hub assembly 520 may be anchored to the bottom of the host pool, the free-floating buoyant configuration is preferred given the resulting insensitivity to tidal or other water level changes.

In a preferred mode of operation of the cultivator (100, 500), the density of the microbiological culture within the cultivator should be slightly lower than that of the host pool. This may be readily achieved by operating a freshwater or brackish culture on ocean water or a saline sea. In one possible mode of operation of the invention, CO₂-bearing gas is added into the system as needed based on measurement of dissolved CO₂ or O₂ concentration. In some embodiments, the phototrophic microorganism culture is maintained at a density in a range of about 0.1 g to about 4.0 g of biomass on dry weight basis per liter of culture. There is no mixing between the culture within the cultivator and the host body of water upon which the cultivator is floating.

In another possible mode, gas is recirculated from the interior of the bioreactor to increase the percentage of CO₂ uptake prior to venting. In a preferred embodiment, gas containing CO₂ is introduced upstream of the circulation pump at a point where the fluid pressure is below 1 Atm., such that additional equipment for pumping the gas is not required. In another possible embodiment, pumped culture is passed through a venturi device to achieve the same effect.

In yet another embodiment, mixing wands 120 could be rotated using a mechanical drive attached to frame 400 and the end of wands 120 to provide mixing through a means other than that caused by the fluid passing through wands 120.

It should be noted that since the energy cost required to operate the mixing and injection system may represent a significant operating cost, in some embodiments it is beneficial to modulate the rate of pumping to match the biological requirements of the species, for instance by reducing flow at night or during cloudy weather. The pumps and air exchange blowers/fans can be powered using energy from renewable resources, e.g., biofuels; solar, wind or wave energy, or can utilize traditional energy sources (e.g., electricity from a grid, carbon-based fuels). The same AC supply can power both the recirculating pump and the blowers/fans. For example, motors driving the pumps or blowers/fans can be powered with solar panels, and power can be distributed via underwater cables. Alternatively, local power could be provided to a collection of cultivators with a floating generator (e.g., a GMI 1011 series—Deutz diesel generator) or a microturbine (e.g., by Capstone; on the internet at capstoneturbine.com/). In some embodiments, the pumps run continuously, i.e., 24 hrs per day. Because algae mixing is most required when sunlight is present, in some embodiments, the pumps and/or fans run during daylight hours. For example, mixing can be controlled by a light (PAR) sensor or direct channeling of energy generated by solar panels. Also, the rate of mixing can be adjusted to correspond to the amount of light available for growth: the flow rate of culture fluid recirculation and therefore the amount of mixing by the mixing wands can be increased with increasing availability of solar radiation available for growth.

It should be noted that under some conditions it may be beneficial to operate the mixing system in pulsatile mode to provide vigorous episodic mixing while conserving pumping energy. Since mixing requirements are generally correlated with solar energy flux, it may be beneficial to operate the pump partially or entirely on electricity generated by photovoltaic modules. In one possible embodiment of the invention, an oxygen permeable membrane may be integrated into the reactor design to preferentially allow the release of oxygen and nitrogen from the cultivator while preventing release of CO₂.

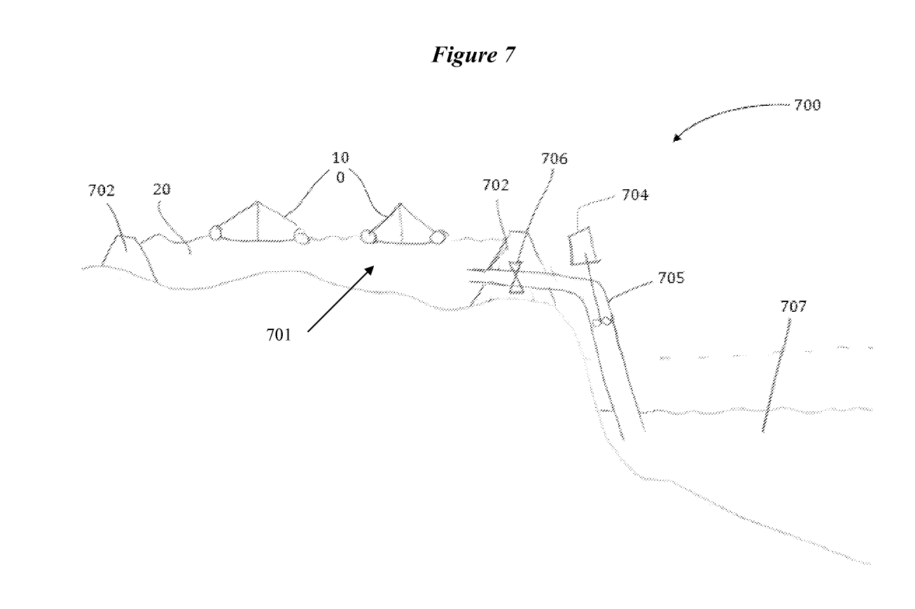

In some shoreside applications of the cultivator of the present invention is located in proximity to a cold water reservoir where a host pool must be fabricated (for example by providing a dam or lined pit with bermed margins), shoreside artificial pool, as illustrated in FIG. 7, may be fabricated. Pool 701, enclosed by berms 702, hosts cultivator modules 100 forming cultivator system 700. Bidirectional pump/turbine 704, as are well known in the art for pumped hydroelectric storage, may be used in conjunction with fluid conduit 705 and valve 706 to withdraw and return cold ocean water 707 as needed to maintain optimal growth temperatures.

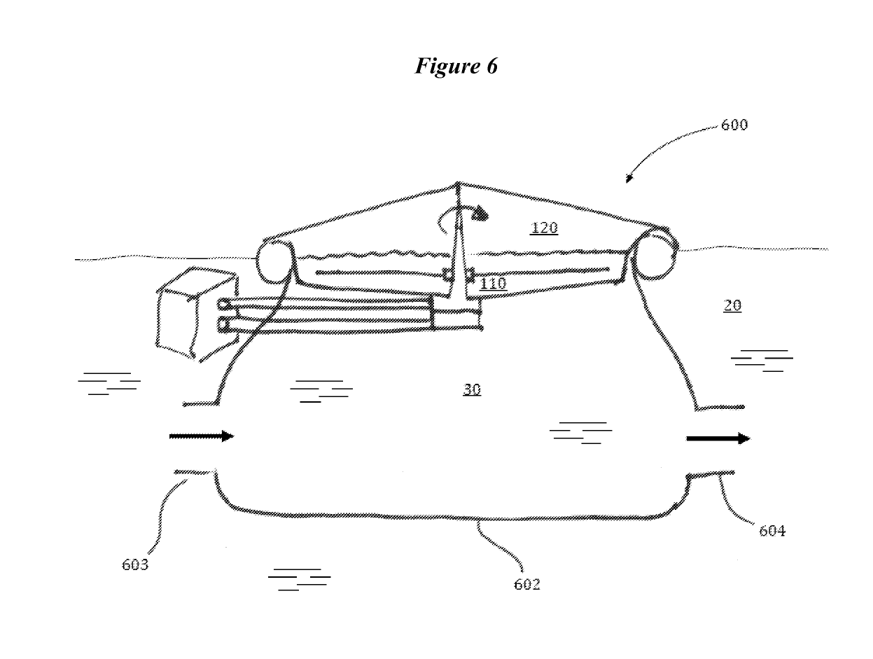

In some shoreside applications, the cultivator of the present invention incorporates host pool 30 into the structure of cultivator 600, as illustrated in FIG. 6. An integrated cultivator 600 with a double bottom 602 may be fabricated with inlet port 603 and outlet port 604, for host fluid, providing the thermal and mechanical benefits of the waterborne cultivator 600 with reduced site preparation cost, and improved access for maintenance. Manufacturing technology capable of producing this type of structure has become well established in recent years for the production of soft-wall aboveground swimming pools. To scale the installation, cultivators 600 are connected with the host fluid volumes connected fluidically in series, and host fluid would be slowly pumped through cultivator 600 at a rate dictated by thermal considerations.

Photosynthetic organisms are very specific with respect to the spectral bands which are absorbed, and large portions of the spectrum are not required. Accordingly in some configurations of the present invention, superior growth conditions may be achieved by employing a pigmented or otherwise spectrally specific top sheet. In some cases dye-based thin film photovoltaic cells may be incorporated into the top sheet to produce electricity simultaneously with biomass, making efficient use of the entire solar spectrum. Electricity so generated may be used to drive the mixing system, or delivered to the electric grid.

In some variations of the present invention, a single cultivator includes multiple mixing assemblies 200, each coupled with support system 300, which includes a pump 310 and CO₂ source 350.

It should be noted that the necessary algae culture conditions for large-scale systems have been determined through the long-term operation of raceways and circular ponds, although laboratory and theoretical productivities are rarely met due to both practical and economic considerations affecting the design and operation of these systems. The major conditions required for the growth of photosynthetic algae include; availability of light (available photons for photosynthesis), maintenance of a temperature range within the healthy range required by the given algae species, availability of CO₂ for photosynthesis, maintenance of a pH range within the healthy range of the given algae species, availability of chemical nutrients for growth of the algae, and a method for the removal (passive or active) of the O₂ generated during photosynthesis.

The present invention provides for the maintenance of the aforementioned conditions, and allows for the conditions to be manipulated and thereby meet the specific needs of various species of photosynthetic microalgae. The term “microalgae” refers to microphytes, e.g., unicellular eukaryotic species that exist individually or in chains or groups. The microalgae subject to the present concentrating methods generally have an average diameter of about 20 μm or less, for example, about 15 μm, 10 μm, 5 μm, or less. In some embodiments, the microalgae are photosynthetic algae. In some embodiments, the microalgae are of the genus Dunaliella, Chlorella, Tetraselmis, Botryococcus, Haematococcus, Phaeodactylum, Skeletonema, Chaetoceros, Isochrysis, Selenestrum, Scenedesmus, Nannochloropsis, Nannochloris, Pavlova, Nitzschia, Pleurochrysis, Chlamydomas or Synechocystis.

The species of algae that may be grown in cultivators, according to the present invention, but are not limited to members of the orders, are: Nannochloropsis including Nannochloropsis oculata and Nannochloropsis salina, Isochrysis including Isochrysis galbana, Chlorella including Chlorella minutisima and Chlorella vulgaris, Scenedesmus including Scenedesmus quadricauda and Scenedesmus obliquus, Haematococcus including Haematococcus pluvialis and Haematococcus lacustris, and Dunaliella including Dunaliella salina and Dunaliella tertiolecta, Selenastrum including Selenastrum minutum, Tetraselmis including Tetraselmis chuii and Tetraselmis suecica. The cultivators find use in culturing a homogenous population of photosynthetic micro-organisms, e.g., a population that is substantially all the same species of photosynthetic micro-organism, e.g., that is at least about 95%, 96%, 97%, 98%, 99% the same species of photosynthetic micro-organism.

These species require the maintenance within a 10-20 degree C. temperature range to grow optimally, which is readily provided by selecting a host pool of the appropriate temperature. The floating photosynthetic cultivators can maintain an aquatic microalgae culture at temperatures suitable for culture of cold water microalgae species, e.g., including without limitation Selenestrum, Scenedesmus, Nannochloropsis or Isochrysis. Generally, the temperature of the aquatic microalgae culture is maintained at temperatures that are at or below about 35° C. In some embodiments, the aquatic microalgae culture is maintained at a temperature in the range of about 15° C. to about 35° C., for example, from 20-35° C., 15-25° C. or 15-30° C. In some embodiments, the aquatic microalgae culture is maintained at a temperature of about 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., or higher or lower, depending on the temperature of the host water pool, as well as its depth and currents (i.e., circulation of water in the host pool).

Additionally, for most microalgae species, the pH of the liquid culture preferably is maintained within a range of 6-8 while providing enough CO₂ for photosynthesis. For a particular species, the preferred pH can be determined in the laboratory cultures. Once that is determined the present cultivator systems can maintain that pH within the range of about 1 pH unit or less around (i.e., above or below) the preferred pH value, for example, within about 0.8, 0.6, 0.4 or 0.2 pH units around (i.e., above or below) the preferred pH value. For example, in cultivating a photosynthetic microalgae species with a preferred culture fluid pH of about 7.0, the present cultivator systems can maintain the culture fluid at a pH in the range of from about 6-8 (i.e., within about 1 pH unit around the preferred pH value), and more preferably in the range of about 6.8-7.2 (i.e., within about 0.2 pH units around the preferred pH value). In the present cultivator systems, the pumps are in fluid communication with an external source of CO₂ delivery and a pH monitoring unit. CO₂ is introduced into the recirculating culture on the suction side of the pump. pH can be monitored upstream of injection and downstream of injection of the CO₂. The pH can be monitored using any method known in the art. pH monitoring units are commercially available. For example, a Signet model 2764-00 probe with a Signet model 3-2750-2 signal conditioner finds use. The pH upstream and/or downstream of the CO₂ injection site is monitored and used to regulate the injection. These signals are wired into a custom controller that is capable of accepted upper and lower pH limits and a hysteresis value. A National Instrument cRIO processor also finds use to perform this regulation. A solenoid valve (e.g., Asco Red Hat) is opened or closed to admit CO₂ into the fluid stream based on the actual pH relative to the target pH of the recirculating culture fluid. If the pH of the culture fluid is determined to be above the target pH, then CO₂ is added, or increased levels of CO₂ are added, to the culture fluid to reduce the pH (i.e., through the formation of carbon acid). If the pH of the culture fluid is determined to be below the target pH, then CO₂ is not added or reduced amounts of CO₂ is added to the recirculating culture fluid. In the presence of sun and photosynthetic activity, the CO₂ is consumed by the algae and the pH increases. The present photosynthetic cultivators utilize an on-off control scheme to keep the pH between these limits.

For marine species, medium can be made from filtered seawater or sea salt must be added at 35 g/L to fresh water. Finally, specific combinations of chemical macro and micro-nutrients must be provided. The nutrient profiles vary with species but macronutrients generally include nitrogen in the form of NaNO₃ at 1.5 g/L, and phosphorous in the form of K₂HPO₄ at 0.04 g/L. Micronutrients required for growth are needed at much lower concentrations and generally include FeSO₄*7H₂O at 0.01 g/L, ZnSO₄*7H₂O at 0.222 ug/L, MnCL₂*4H₂O at 1.81 ug/L, CuCl₂*2H₂O at 0.08 ug/L, CoCL₂*6H₂O at 0.049 g/L, and Na₂MoO₄*2H₂O at 0.391 ug/L.

In some embodiments, the cultivator further comprises a processing unit that monitors the conditions (e.g., pH, temperature, density of photosynthetic micro-organisms, salinity, levels of CO₂ and O₂, etc.) of the culture fluid. For example, the culture fluid can be drawn up from the cultivation space in the cultivator and passed through the processing unit and then returned to the cultivation space through the central hub assembly and the mixing wands. Gas levels (e.g., CO₂), micronutrients, salt levels, pH, temperature, and other monitored growth parameters can be adjusted as needed as the culture fluid is returned to the cultivation space. This can be accomplished by continuous or intermittent cycling of the culture fluid, as desired.

Assuming that all of the previously described conditions are maintained within the ranges generally outlined in scientific literature for each given species, and that the outlined chemical nutrients are provided, the two factors that are often most limiting to productivity are availability and use of light and the delivery of sufficient CO₂. The photosynthetic cultivator systems and modules (100, 500, 600 and 700) of the present invention improves both light usage and CO₂ delivery by injection of CO₂ throughout the growth area, and concurrently, as a result of injecting culture that has been in intimate contact with CO₂ for a significant amount of time in the form of jets throughout the growth area, a great amount of turbulence is created. This vertical mixing (i.e., photosynthetic micro-organisms that have settled to the bottom of the culture fluid are disrupted and mixed into the culture fluid) created by and with culture that has a high amount of available CO₂ available, allows for the vertical passage of individual algae cells into and out of the light on the surface of the culture. In so doing, the photosynthetically available radiation is effectively diluted among many more individual algae cells getting quick bursts of light, with the added benefit of providing CO₂ to the culture at the same location where this efficient mixing and utilization of light is occurring. Although the raceway and circular ponds can provide the same type of turbulent mixing, they are often CO₂ limited due to the introduction of CO₂ at a single point source and the turbulent mixing decreases as the culture flows through the raceway.

In addition to increasing vertical mixing, another way to more efficiently utilize light is to grow the culture to higher biomass densities by providing the same amount of light and mixing to a culture of shallower depth. In the present invention the depth can easily be manipulated to match the target culture concentration. Increased biomass density enables use of more economical biomass separation hardware since the volumes and flows required to process a given quantity of biomass are decreased.

### EXAMPLES

The following examples are offered to illustrate, but not to limit the claimed invention.

### Example 1

### Exemplified Embodiment of Floating Cultivator for Phototrophic Organisms

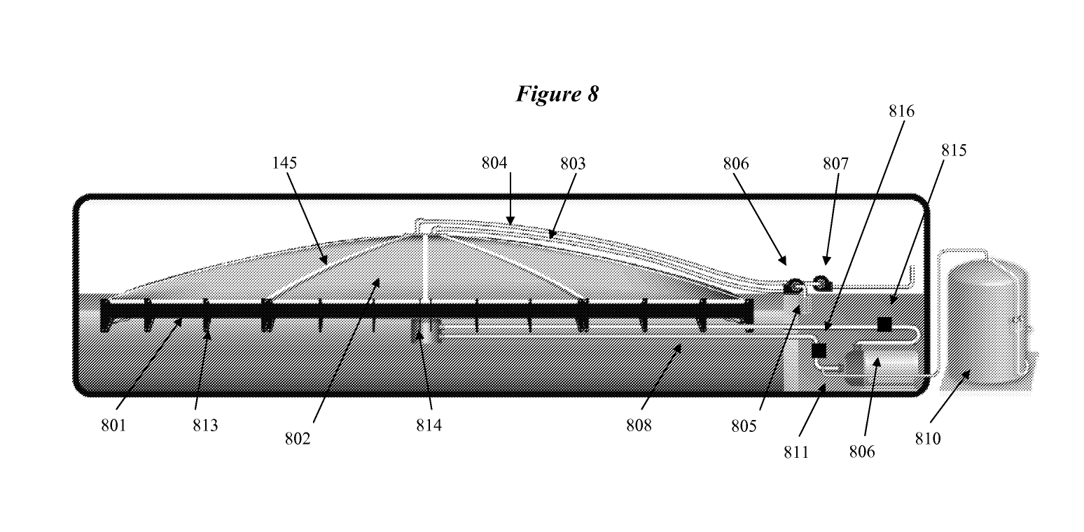

FIGS. 8-11 illustrate an embodiment of the floating cultivator floating in an above-ground pool. The cultivator is comprised of a floating ring with a central mast and flexible bottom and top layers. The floating cultivator is depicted as the ring (801), upper canopy (802), lower canopy (not visible in drawing), and central mast (not visible in drawing). Upper pipes (or tubes) are for air supply into (803) and out of (804) the upper canopy. Fresh air is taken in through an inlet air filter (805). The flow of fresh air into the cultivator is facilitated by an inlet blower or fan (806); the flow of oxygen-rich air out of the cultivator is facilitated by an outlet blower or fan (807). Fluid lines (e.g., pipes or tubes) below (808) go to a recirculating pump (809). The air lines and the culture fluid lines may be rigid or flexible depending on what is appropriate for a particular installation and the expected motion of the cultivator based on environmental conditions (wind, currents, waves, etc.).

The central mast can be attached to the ring both top and bottom via flexible fabric straps (145) with ratchet mechanisms (813). In this embodiment, a total of sixteen straps are used: eight on the top side and eight on the bottom side. By adjusting the differential tension of the straps, the orientation and vertical positioning of the mast can be controlled. The center mast comprises a rotary union that distributes recirculating culture to the fluid distribution arms attached to the center mast. These arms possess several holes along the length which form jet when supplied with pressurized culture. The jets cause the arm to rotate. By alternating the pressure and flow of the culture being supplied to the arms and the orientation of the jets relative to the culture surface, the rotational speed of the arm can be adjusted. These factors are adjusted to nominally have linear velocity at the arm tip of about 30 cm/sec. The rotation of the arms facilitates mixing and enables uniform distribution of dissolved CO₂ to the cultivator. In this embodiment, the motor driving the pump is the only motor driving the recirculation of the culture fluid throughout the system.

There is a return sump at the base of the mast and located inside the lower layer (static distribution manifold 814 depicted). Culture flows into and out of the cultivator from the lower part of the mast. The culture is recirculated by a pump system. A vacuum aspirator can draw CO₂ into this recirculation culture from a CO₂ tank (810). A combination of flow through the centrifugal pump and a static mixer aids in the CO₂ dissolving into the water. pH is measured upstream (815) and downstream (816) of the CO₂ injection point (811). The downstream pH measurement is in communication with an on-off controller with hysteresis to regulate the pH within a range appropriate for the species under cultivation. As more CO₂ is injected into the recirculating culture, the pH is reduced. Photosynthetic activity consumes the CO₂ and causes the pH to rise. Monitoring the pH at the upstream location and trending over time is an indirect measure of photosynthetic activity. In addition to monitoring pH, other parameters can be monitored in the recirculation loop, including, e.g., flow rate, temperature, conductivity, and optical density.

The bottom layer of the cultivator isolates the culture volume from the body of water on which the cultivator floats. The ring is fabricated from buoyant material sufficient to allow the cultivator to float. In this embodiment, eight inch diameter HDPE (High Density Polyethylene) pipe was used. The pipe ring can be filled with foam sealant to help avoid water infiltration in the event that the pipe is punctured. For example, the sealant “Great Stuff” by Dow finds use (on the internet at greatstuff dow.com). The bottom layer can be a polyethylene film that contains the culture. This film can be attached to the ring via a clamp system comprised of two close tolerance pipes that sandwich the film in place. In this embodiment, the inner pipe had a portion of its circumference machined away, and the outer pipe was left intact. The inner pipe inside diameter (IID) and the outer pipes outside diameter (OOD) were selected such that OOD>IID. This provides a clamping force to hold the film in place.

Furthermore, there are several locations around the circumference in which a strap attaches to the outside (non-culture) surface of the lower layer to a ratchet mechanism (813) attached to the ring (801). In the depicted embodiment, 24 ratchets were attached to the ring. This ratchet and strap system can used to maintain the bottom in as flat a state as possible to avoid accumulation of biomass in the vicinity of the ring. In the absence of these straps, the bottom layer may develop a taper toward the outer ring that forms a beach within the culture where biomass tends to accumulate.

The cultivator also has a upper film layer. This isolates the algae culture from the nominal exterior environment. This embodiment depicts an inflated structure. One blower was used to supply external air to the vapor space above the culture. A second blower was used to discharge air from this space. During high photosynthetic activity, algae produce oxygen. This oxygen diffuses from the culture with the benefit of the mixing provided by the rotating arms. The supply and discharge fans for the inflated space about the cultivator limit the amount of oxygen in this vapor space and assure that the oxygen concentration does not build to a level that would otherwise inhibit algae growth.

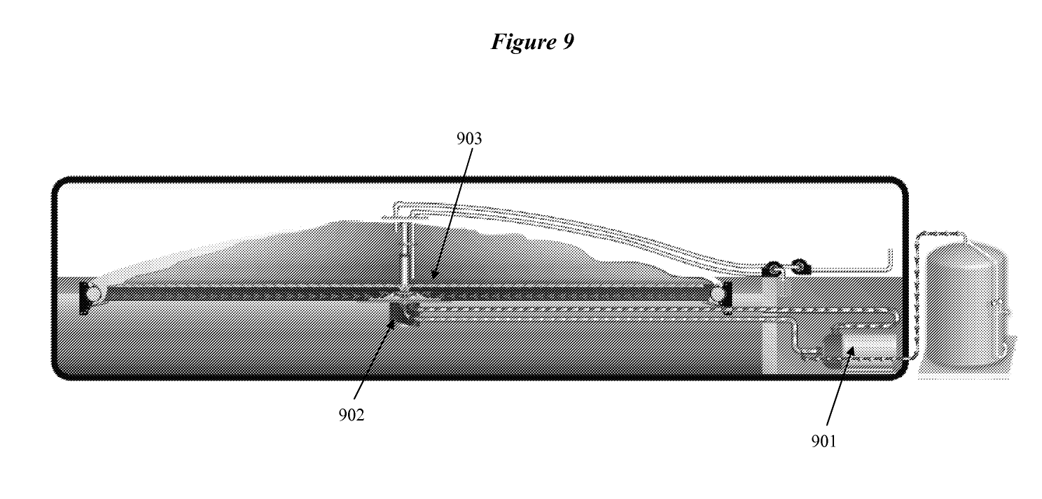

FIG. 9 illustrates the flow path for the culture (depicted as dotted lines). Under nominal operating conditions, the 12,000 L capacity of the 6 m radius cultivator recirculates once every 45 minutes. The pump suction (901) draws from the central mast (902). The discharged culture is distributed from the inside radius to the outer radius by the mixing action of the wands (903).

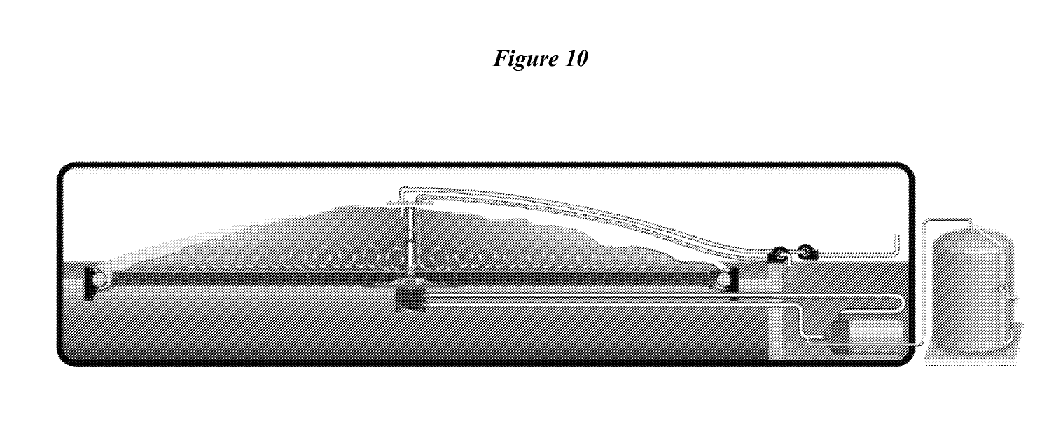

FIG. 10 illustrates the initial inflation of the upper canopy. The air pressure within the canopy is raised sufficiently to stretch the upper film layer. During inflation of the upper canopy, no vapor is discharged from the canopy.

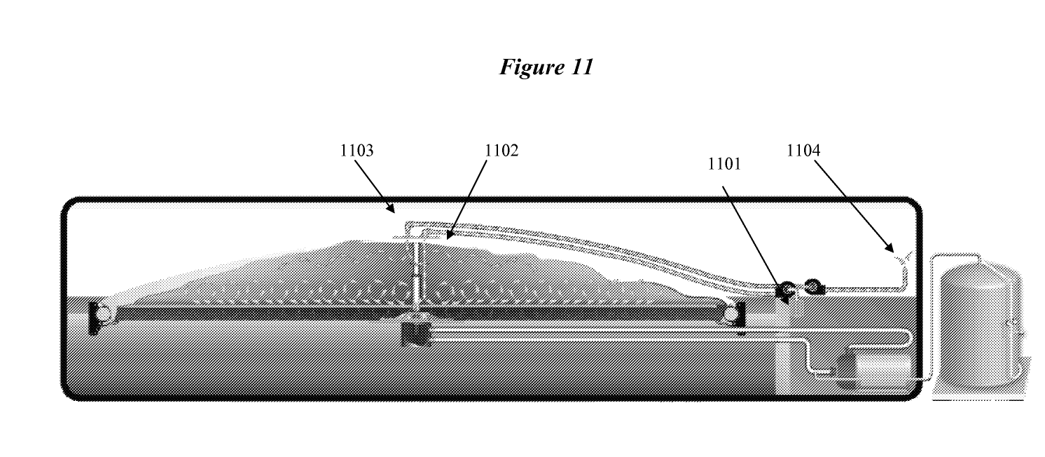

FIG. 11 illustrates a balance between the fresh exterior air supplied to the canopy (1101, 1102) and the air that discharges (1103, 1104), once the canopy is inflated. This air exchange helps limit the amount of oxygen build up. By limiting the rate of exchange, the vapor space above the culture can attain a higher than normal concentration of carbon dioxide. The air exchange aids in limiting CO₂ loss and improving uptake of CO₂ by the algae.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A waterborne cultivator for photosynthetic micro-organisms, comprising:
a buoyant frame connected to a top sheet sufficiently transparent to admit light to support photosynthetic growth of the micro-organisms and a bottom sheet, the top sheet and bottom sheet connected to a central hub assembly in an arrangement to create a cultivation space comprising culture fluid comprising a homogenous population of photosynthetic micro-organisms, the central hub assembly comprised of a vertical hollow cylinder forming a vertical axis, the cylinder having an inlet and one or more outlets in fluid communication with one or more mixing wands extending substantially perpendicular from the cylinder, the mixing wands having one or more or more holes on one side of the wand, wherein the culture fluid is delivered into the inlet and through the holes of the mixing wands, wherein the wands rotate with the central hub assembly around the vertical axis, wherein fluid pumped through the wands is ejected into the culture fluid, thereby promoting turbulent vertical mixing of the photosynthetic micro-organisms.

2. The cultivator of claim 1, wherein the photosynthetic micro-organism is phototrophic algae.

3. The cultivator of claim 1, wherein the bottom sheet and top sheet connected to the frame and the central hub assembly form a liquid-tight enclosure.

4. The cultivator of claim 1, wherein CO₂ is co-delivered into the inlet with the culture fluid.

5. The cultivator of claim 1, wherein the inlet of the cylinder is in fluid communication with a pump that recirculates the culture fluid, wherein the pump draws culture fluid from the cultivation space through a suction line and returns culture fluid to the cultivation space through a pressure line into the inlet and through the holes of the mixing wands.

6. The cultivator of claim 5, further comprising at least one pH monitor in fluid communication with the recirculating culture fluid, wherein the pH monitor is in communication with an external CO₂ source, wherein if the pH of the culture fluid is above a target pH value, CO₂ is injected into the recirculating culture fluid.

7. The cultivator of claim 6, wherein a first pH monitor is positioned upstream of a site for CO₂ injection and a second pH monitor is positioned downstream of the site for CO₂ injection.

8. The cultivator of claim 1, wherein fluid pumped through the mixing wands creates a reaction force which rotates the central hub assembly.

9. The cultivator of claim 1, wherein the inlet of the cylinder is in fluid communication with a motor and impeller integrated into the central hub assembly.

10. The cultivator of claim 1, wherein the central hub assembly is in fluid communication with a processer that monitors the culture fluid conditions.

11. The cultivator of claim 1, wherein the inlet of the cylinder is submerged in the culture fluid.

12. The cultivator of claim 1, wherein the top sheet has an air inlet for intake of air external to the cultivation space and an air outlet for discharge of oxygen-rich air from within the cultivation space.

13. The cultivator of claim 1, wherein the frame is circular.

14. The cultivator of claim 1, wherein the cultivator is floating in a host pool.

15. The cultivator of claim 1, wherein the culture fluid comprises substantially uniformly distributed CO₂.

16. A culture fluid produced in a cultivator of claim 1 comprising a homogenous population of photosynthetic algae cells, wherein the culture fluid comprises substantially uniformly distributed CO₂ and the individual algae cells in the population have been substantially uniformly exposed to light.

17. A method for cultivating photosynthetic microorganisms by culturing the photosynthetic microorganisms in a waterborne cultivator of claim 1, wherein the photosynthetic microorganisms are cultured in a culture fluid comprising substantially uniformly distributed CO₂.

18. The method of claim 17, wherein the cultivator is outside and the photosynthetic microorganisms are grown under natural sunlight.

19. The method of claim 17, wherein the cultivator is floating in a host pool.

20. The method of claim 17, wherein the cultivator is in fluid communication with a pump that recirculates the culture fluid, wherein the pump draws culture fluid from the cultivation space through a suction line and returns culture fluid to the cultivation space through a pressure line into the inlet and through the holes of the mixing wands.

21. The method of claim 20, wherein the cultivator further comprises at least one pH monitor in fluid communication with the recirculating culture fluid, wherein the pH monitor is in communication with an external CO₂ source, wherein if the pH of the culture fluid is above a target pH value, CO₂ is injected into the recirculating culture fluid.
